SEQUENCE LISTING (1 sequence)

SEQ ID NO: 1 - protein, 498 aa, influenza A viral nucleoprotein
MASQGTKRSYEQMETDGERQNATEIRASVGKMIDGIGRFYIQMCTELKLSDYEGRLIQNSLTIERMVLSAFDERRNKYLEEHPSAGKDPKKTGGPIYKRVDGKWMRELVLYDKEEIRRIWRQANNGDDATRGLTHMMIWHSNLNDTTYQRTRALVRTGMDPRMCSLMQGSTLPRRSGAAGAAVKGIGTMVMELIRMIKRGINDRNFWRGENGRKTRSAYERMCNILKGKFQTAAQRAMMDQVRESRNPGNAEIEDLIFSARSALILRGSVAHKSCLPACVYGPAVASGYDFEKEGYSLVGIDPFKLLQNSQVYSLIRPNENPAHKSQLVWMACHSAAFEDLRLLSFIRGTKVSPRGKLSTRGVQIASNENMDTMESSTLELRSRYWAIRTRSGGNTNQQRASAGQISVQPTFSVQRNLPFDKSTIMAAFTGNTEGRTSDMRAEIIRMMEGAKPEEVSFRGRGVFELSDEKATNPIVPSFDMSNEGSYFFGDNAEEYDN

(19) United States
(12) Patent Application Publication
(10) Pub. No.: US 2009/0220938 A1
(43) Pub. Date: Sep. 3, 2009

(54) VIRAL NUCLEOPROTEIN DETECTION USING AN ION CHANNEL SWITCH BIOSENSOR

(76) Inventors: Manoj Kumar, Fremont, CA (US); Sang-Kyu Lee, Palo Alto, CA (US)

Correspondence Address: HOWREY LLP-CA, C/O IP DOCKETING DEPARTMENT, 2941 FAIRVIEW PARK DRIVE, SUITE 200, FALLS CHURCH, VA, 22042-2924, US

(21) Appl. No.: 11/911,325

(22) PCT Filed: Apr. 13, 2006
(86) PCT No.: PCT/US06/14284

§ 371 Date: Jul. 16, 2008

Related U.S. Application Data
(60) Provisional application No. 60/671,945, filed on Apr. 15, 2005.

(51) Int. Cl. C12Q 1/70; C12M 1/00

(52) U.S. Cl. 435/5; 435/287.2

(57) ABSTRACT

The present invention provides a method of detecting viruses, such as respiratory-related viruses, in a sample with a sensitivity of at least 80%, and/or specificity of at least 90%, and/or with an accuracy of at least 90%. The method comprises contacting the sample with a biosensor. The present invention also provides a biosensor comprising a membrane and a solid conducting surface, with the membrane being attached to the solid conducting surface in a manner such that a reservoir exists therebetween. The membrane comprises first and second layers each comprising closely packed amphiphilic molecules; a plurality of first and second ionophores located in the first and second layers, respectively; and a plurality of antibodies or fragments thereof directed against nucleoproteins of respiratory-related viruses, more specifically, nucleoproteins of an influenza virus, and covalently attached to the second ionophores. The present invention further provides a device comprising an array of such biosensors.

### BACKGROUND OF THE INVENTION

1. Field of the Invention

The present invention relates to methods of detecting a viral nucleoprotein with high sensitivity, specificity and/or accuracy using a biosensor. The present invention also relates to a biosensor comprising a plurality of antibodies or fragments thereof as receptor molecules which recognize and are capable of binding to a viral protein. The present invention further relates to a device comprising an array of such biosensors.

2. Description of the Related Art

The properties of ion channels have been exploited for construction of the Ion Channel Switch (ICS) biosensor (Cornell, et al., Nature 387, 580-3 (1997)). The ICS biosensor technology, pioneered by Dr. Bruce Cornell and his colleagues at Ambri Ltd. (Chatswood, Australia), utilizes a novel transduction mechanism based on an ion channel-containing biomimetic membrane that may readily be adapted to detect a wide range of biological agents.

Ion channels are membrane protein complexes that play an essential role in the diffusion of ions across cell membranes. The phospholipid bilayers that form biological membranes are known to produce a hydrophobic, low dielectric barrier to hydrophilic and charged molecules. Transport of ions across biological membranes is a ubiquitous mechanism for physiological processes such as nerve impulse propagation (Av-Ron & Rospars, Biosystems 36, 101-8 (1995)). A well-documented example is the flux of cations triggered by acetylcholine in the acetylcholine receptor channel present in cross-synaptic nerves (Reiken, et al., Biosensors and Bioelectronics 11, 91-102 (1996)). An important feature of this process is the amplification of the recognition event, whereby detection of a single molecule could trigger the passage of up to 106 ions per second across an otherwise electrically impermeable membrane.

A simple and well-studied example of an ion channel is the polypeptide gramicidin (gA), a naturally occurring antibiotic (Woolley & Wallace, J Membr Biol 129, 109-36 (1992)). When incorporated into a lipid bilayer membrane, it forms electrically conductive channels. A current of ions across the membrane is “switched on” by the dimerization or alignment of gramicidin monomers diffusing within the two leaflets of the lipid bilayer. The lifetime of this channel-forming event is of the order of 1 second. Once the channel is formed it permits the flux of small monovalent cations at maximum rates of 10⁶-10⁷ ions/sec. For these reasons, gramicidin makes an ideal bio-electronic switch for a biosensor.

To build the ICS biosensor, gramicidin is incorporated into a biomimetic membrane built with phospholipids resembling those that are encountered in highly stable cell membranes of extremophilic microorganisms (see FIG. 1, panel A). The lipid bilayer membrane is stabilized by tethering the lipids to a gold electrode, by means of thiol chemistry and an intervening hydrophilic linker to create a reservoir for ions at the electrode surface (Knoll, et al., J Biotechnol 74, 137-58 (2000); and Krishna, et al., Langmuir 17, 4858-4866 (2001)). The immuno-sensing based detection is achieved by attaching antibody fragments to the mobile outer layer gramicidin channels. Complementary antibody fragments are also attached to stationary membrane-spanning lipids that are tethered to the gold electrode. When an analyte is captured between the two antibody fragments, the mobile gramicidin channels of the outer leaflet are thereby anchored to the stationary lipids (see FIG. 1, panel C, ‘Gated closed’) preventing the formation of conductive dimeric channels since the inner leaflet gramicidin molecules are also tethered to the gold surface (see FIG. 1, panel B, ‘Gated open’). The reduction in number of total available gramicidin dimers results in a rapid decrease in current across the membrane. This switching mechanism provides the means for the translation of a single biological event (e.g., the binding of analyte to a pair of analyte-recognizing antibody fragments) into a significantly amplified electrical signal (e.g., a change of flux of 10⁶ ions/sec per channel). Such degree of amplification can be used in creating a sensitive assay platform.

The ICS has all the required elements for detection and signal amplification incorporated within the tethered membrane, and therefore there is no need for washing or equilibration steps. The gating of ion channels resulting from analyte capture is such that the rate of decrease in current across the membrane is directly proportional to analyte concentration; the dose response obtained with ICS has proved to be linear over a wide dynamic range. This detection mechanism gives reliable quantitative results for the detection of a variety of analytes (Cornell, et al., Optical Biosensors: Present and Future (eds. F., L. & C., R. T.) 457 (Elsevier, Amsterdam, 2002).

General biosensor and membrane technology and particularly ion channel switch (ICS) biosensors are described in U.S. Pat. Nos. 5,874,316, 5,234,566; 5,443,955; 5,741,409, 5,401,378; 5,637,201; 5,753,093; 5,783,054; 6,316,273; 6,451,196; 6,573,109; and 5,741,712, as well as in the published PCT application WO 98/55853; the contents of which are incorporated herein by reference.

Concerns about the spread of infectious diseases and threat of biological warfare and terrorism have accelerated the need for low-cost, portable biodetection technologies that can rapidly and reliably detect one or more pathogens from a single environmental or human body fluid sample. Timely identification of these fast-acting pathogens is critical, but difficult to implement with the current diagnostic tools used in public health and hospital based clinical laboratories. It is therefore desirable that primary care settings rely on a highly sensitive, specific, inexpensive, and easy-to-use detection method and/or system that could rapidly and accurately identify a pathogenic virus.

One example of the fast-acting pathogens is influenza virus. Several clinical diagnostic kits and central lab methods based on qualitative and quantitative immunochromatogenic detection methods for influenza virus are currently available (Uyeki, Pediatr. Infect Dis J., 22, 164-77 (2003)). These detection kits or methods either detect nucleoprotein antigens or neuramidase enzyme of the influenza virus. However, their sensitivity is dependent on the colorometric detection method and tends to be unsatisfactory. For example, Directigen Flu A Kit for detection of influenza A and B viruses has an overall sensitivity of 43.83%, making the Kit a less accurate screening test for large populations (Cazacu, et al., J. Clinical Microbiology, 42(8), 3707-3710, (2004)). There is a need for highly sensitive, specific and accurate method of detecting viruses, and specifically respiratory-related viruses such as influenza viruses.

### SUMMARY OF THE INVENTION

The present invention is directed to a method of detecting viruses, such as respiratory-related viruses, and more specifically, an influenza virus, in a sample with a sensitivity of at least 80%, and/or a specificity of at least 90%, and/or an accuracy of at least 90% by contacting the sample with a biosensor.

The biosensor suitable for this invention comprises a membrane and a solid conducting surface, wherein the membrane is attached to the solid conducting surface in a manner such that a reservoir exists therebetween. The membrane advantageously comprises first and second layers each comprising closely packed amphiphilic molecules; a plurality of first and second ionophores located in the first and second layers, respectively; and a plurality of antibodies or fragments thereof covalently attached to the second ionophores.

The antibodies or fragments thereof in the present invention are directed against and capable of binding to nucleoproteins of respiratory-related viruses, more specifically, nucleoproteins of an influenza virus. Preferably, the influenza virus is an influenza A virus and the antibodies and fragments thereof are monoclonal antibodies or fragments thereof directed against influenza A virus.

Ionophores include gramicidin (preferably gramicidin A), band three protein, bacteriorhodopsin, proteorhodopsin, mellitin, alamethicin, an alamethicin analogue, porin, tyrocidine, tyrothricin, and valinomycin. In one embodiment, dimeric ionophores such as gramicidin are used in the present invention. The first ionophores are prevented from lateral diffusion in the first layer; and the second ionophores are capable of lateral diffusion within the second layer. The binding of the antibodies or fragments thereof to the influenza viral nucleoprotein causes a change in the relationship between the first and the second ionophores such that the flow of the ions across the membrane via the first and second ionophores is prevented. In this method, reduction of admittance of the membrane corresponds to the presence of an influenza viral nucleoprotein.

The present invention has several advantages over the existing detection methodologies. First, ICS is based on electronic transduction of a biological recognition event, lending itself to low cost instrumentation and inexpensive microarray chip technology. Second, sample preparation for ICS is often unnecessary in the case of biological fluids such as saliva or blood, and the analysis is typically completed in less than 15 minutes.

The present invention further provides a biosensor. Such biosensor comprises a membrane and a solid conducting surface, wherein the membrane is attached to the solid conducting surface in a manner such that a reservoir exists therebetween. The membrane comprises first and second layers each comprising closely packed amphiphilic molecules; a plurality of first and second ionophores located in the first and second layers, respectively; and a plurality of antibodies or fragments thereof covalently attached to the second ionophores. The antibodies or fragments thereof are capable of binding to nucleoproteins of respiratory-related viruses, more specifically, nucleoproteins of an influenza virus. The biosensor has at least 80% sensitivity and/or at least 90% specificity and/or at least 90% accuracy when used to detect an influenza viral nucleoprotein in a sample.

These and other objects will be more readily understood upon consideration of the following detailed descriptions of embodiments of the invention.

### BRIEF DESCRIPTION OF THE DRAWINGS

FIG. 1 is a scheme illustrating an ion channel switch (ICS) direct assay system. Panel A: components of the system; panel B: conducting membrane with modeled circuit diagram showing open gramicidin channel; panel C: conducting membrane with modeled circuit diagram showing closed gramicidin channel.

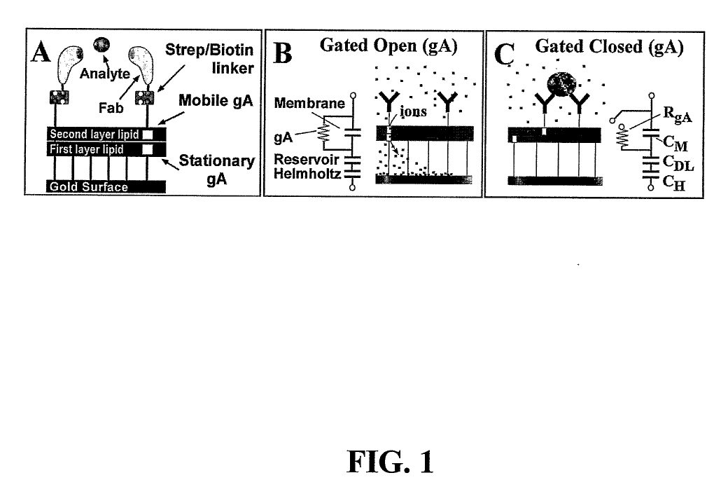

FIG. 2 shows the sequence of influenza A viral nucleoprotein (SEQ ID NO:1).

FIG. 3 is a scheme illustrating influenza A virus and major components.

FIG. 4 illustrates a handheld ICS biosensor. Panel A: universal array chip reader; panel B: disposable biosensor cartridge incorporating microfluidics and on-chip electronics; panel C: actual 4×4 sensor element of ICS microarray chip, mounted with interconnects within the proposed disposable biosensor cartridge.

FIG. 5 illustrates performance characteristics of the influenza A virus antigen test. Panel A: a typical time course of admittance changes upon addition of analyte; panel B: dose response curve; panel C: inverse regression; panel D: Receiver Operating Characteristic (ROC) curve.

FIG. 6 illustrates comparison of ICS vs. ELISA method.

FIG. 7 illustrates the Beckton-Dickinson (BD) test strip (panel A) and ICS test (panel B) results for influenza A test. Top view of Panel A shows a series of images of BD test strip results with different dilutions. Bottom view of Panel A shows signal ratio of BD test image analysis at different concentrations.

FIG. 8 illustrates ROC curve analysis of the ICS™ FluA test.

### DETAILED DESCRIPTION OF THE INVENTION

In order to provide a clear and consistent understanding of the specification and claims, including the scope given to such terms, the following definitions are provided:

As used herein, the term “admittance” refers to an electrical term used to describe the ability of ions to transverse a system when a potential is applied, and is expressed as units of Siemen (S) or Mho (inverse of Ohm). Admittance is the reciprocal of impedance.

As used herein, the term “impedance” is a general expression applied to any electrical entity that impedes the flow of ions. Impedance is used to denote a resistance, a reactance or a combination of both reactance and resistance, with units of Ohm (Ω).

As used herein, the term “an amphiphilic molecule” refers to a molecule having a hydrophilic head portion and one or more hydrophobic tails.

As used herein, the terms “a receptor molecule”, “a capture molecule” and “a recognition molecule” are interchangeable. Each term refers to a molecule that contains a recognition moiety that can bind with some specificity to a desired analyte (target molecule).

As used herein, the term “an antibody fragment” is part of an antibody that contains at least one antigen-binding site and is capable of binding to the antigen. Preferred antibody fragments include fragment antigen binding Fab′ and F(ab′)₂.

As used herein, the term “phase” refers to the delay between applying a voltage and measuring the current in an electrical circuit.

As used herein, the term “reactance” refers to the property of resisting or impeding the flow of ions (AC current or AC voltage) in inductors and capacitors, with units of Ohm (Ω).

As used herein, the term “ionophores” refer to natural or synthetic substances that promote the passage of ions through lipid barriers in natural or artificial membranes. Ionophores may form ion-conducting pores in membranes.

As used herein, the term “accuracy” is determined by estimating the area under the Receiver Operating Characteristic (ROC) curve using trapezoidal rule (Hanley and McNeil, Radiology, 143:29-36 (1982)), as well as described on University of Nebraska Medical Center, Department of Internal Medicine website (http://gim.unmc.edu/dxtests/roc3.htm). An area of 1 represents a perfect test; an area of 0.5 represents a worthless test. A rough guide for classifying the accuracy of a diagnostic test is the traditional academic point system: 0.90-1=excellent; 0.80-0.90=good; 0.70-0.80=fair; 0.60-0.70=poor; and 0.50-0.60=fail.

As used herein, the term “sensitivity” (often referred to as the “true positive rate”) is defined as the number of positive decisions/the number of actually positive cases, whereas the “false positive rate” is defined as the number of negative decisions/the number of actually negative cases (Park and Goo, Korean J Radiol 5(1), 11-8 (2004)).

True Condition Status

| Test Result | Positive | Negative |
|---|---|---|
| Positive | True Positive (TP) | False Positive (FP) |
| Negative | False Negative (FN) | True Negative (TN) |

More formally, the term “sensitivity” can be defined as probability of correctly reporting positives from diseased population. Sensitivity, often expressed as a percentage, can be obtained from the following equation:

Sensitivity=TP/(TP+FN)

As used herein, the term “specificity” is defined as probability of correctly reporting negatives from non-diseased population. Specificity, often expressed as a percentage, can be obtained from the following equation:

Specificity=TN/(TN+FP)

The term “false positive rate” is defined as probability of incorrectly reporting positive from non-diseased population. False positive rate, often expressed as a percentage, can be obtained from the following equation:

False positive rate=1−specificity=FP/(TN+FP)

The term “positive predictive value” is defined as probability of correct prediction of positive test results. Positive predicative value, often expressed as a percentage, can be obtained from the following equation:

Positive predicative value=TP/(TP+FP)

As used herein, the term “cutoff level” refers to an analyte concentration that above which gives a positive test result and below which gives a negative test result.

As used herein, the term “detection limit” refers to the lowest amount (e.g. concentration) of an analyte in a sample for which there is at least a 95% confidence that the concentration of the analyte is greater than zero.

The present invention is directed to a method of detecting viruses, such as respiratory-related viruses, and more specifically, an influenza virus, in a sample with a sensitivity of at least 80%, and/or a specificity of at least 90%, and/or an accuracy of at least 90% by contacting the sample with a biosensor.

In one example, the present method has a detection limit of 0.4 μg/ml of Fitzgerald protein. Fitzgerald protein is an artificial unit, which is the total protein of whole cell sample of influenza A antigen obtained from Fitzgerald (Concord, Mass.), catolog No. 30-A150, lot No. A04080601. Fitzgerald protein contains high concentration of viral antigen (influenza A viral nucleoprotein) and egg proteins. A skilled person can calculate the percentage of pure influenza A viral nucleoprotein in the total protein of whole cell sample, and convert the Fitzgerald protein unit into the pure influenza A viral nucleoprotein unit, if desired.

In some embodiments, the detection limit is at least 1.5, 2.0, 2.5, 3.0, 3.5, 4.0, 4.5, 5.0 times lower (better) than prior art detection methods, such as ELISA, when same amount of antibodies or fragments thereof are used.

In another example, the present method has a detection limit of 0.5 μg/ml of Beckton-Dickinson influenza A virus protein.

The present invention is also directed to a biosensor. The present invention is further directed to a device comprising an array of such biosensors.

The biosensor suitable for this invention comprises a membrane and a solid conducting surface, wherein the membrane is attached to the solid conducting surface in a manner such that a reservoir exists therebetween. The membrane comprises first and second layers each comprising closely packed amphiphilic molecules; a plurality of first and second ionophores located in the first and second layers, respectively; and a plurality of antibodies or fragments thereof covalently attached to the second ionophores. Preferably, the amphiphilic molecules of the second layer comprise phospholipids.

Ionophores include gramicidin (preferably gramicidin A), band three protein, bacteriorhodopsin, proteorhodopsin, mellitin, alamethicin, an alamethicin analogue, porin, tyrocidine, tyrothricin, and valinomycin. In one embodiment, dimeric ionophores such as gramicidin are used in the present invention. The first ionophores in the present biosensor are prevented from lateral diffusion in the first layer; and the second ionophores are capable of lateral diffusion within the second layer. The binding of the antibodies or fragments thereof to nucleoproteins of respiratory-related viruses, more specifically, nucleoproteins of an influenza virus causes a change in the relationship between the first and the second ionophores such that the flow of the ions across the membrane via the first and second ionophores is prevented. In this method, reduction of admittance of the membrane corresponds to the presence of respiratory-related viruses, more specifically, an influenza virus.

The manufacture of the sensor component of the ICS system is simple and takes advantage of self-assembling membranes in the nanofabrication of the biosensor. The membrane is “self-assembled” on top of a gold electrode using a combination of sulfur-gold chemistry and physisorption. The tethered inner leaflet is formed by the deposition of an ethanolic solution of sulfur-containing amphiphilic lipids and gramicidin using a sulfur gold interaction. It provides the hydrophobic surface upon which the second, mobile leaflet of the membrane self-assembles in aqueous buffer (Raguse, et al., Langmuir 14, 648-659 (1998)).

In one embodiment, the outer leaflet of the membrane contains biotin modified gramicidin monomers. This allows the linkage of biotinylated antibody fragments by using biotin-streptavidin interactions. Additionally, a membrane-spanning lipid containing a biotin moiety is directly attached to the gold electrode. Thus, a full ICS biosensor can be assembled by simply adding an aqueous solution of streptavidin followed by the addition of biotinylated antibody fragments specific for the analyte of interest. This allows for a highly automated, reproducible and scaleable manufacturing process.

Samples, which contain an analyte to be detected by the present method, include body samples and non-body samples. Examples of body samples include blood, serum, sweat, tears, urine, saliva, throat swabs, nasopharyngeal aspirates, smears, bile, gastrointestinal secretions, lymph, organ aspirates, and biopsies. These samples can be whole cell samples. Non-body samples include any solution samples not derived from a human body, for example, culture medium, water, saline, organic acids, buffers, soil, food, beverages, powders, building and room surfaces.

The methods described above can be used in general to detect viruses, especially respiratory-related viruses. Examples of respiratory-related viruses that can be detected and quantitated by the present invention include Paramyxoviruses (e.g. respiratory syncytial virus (RSV), parainfluenza), Coronaviruses (e.g. corona) and Orthomyyoviruses (e.g. influenza). A preferred example of influenza virus is influenza A virus.

Furthermore, the present methods can also be used to detect biodefense-related viruses. Examples of biodefense-related viruses that can be detected and quantitated by the present invention include Category A and Category C viruses. Category A viruses include Arenaviruses (e.g. Lassa fever, Junin, Machupo), Bunyaviruses (e.g. Hantaviruses), Flaviruses (e.g. Dengue), and Filoviruses (e.g. Ebola, Marburg). Category C viruses include Rhabdoviruses (e.g. Rabies), Coronaviruses (e.g. Corona, SARS-CoV), and Orthomyxoviruses (e.g. Influenza).

Still furthermore, the present methods can also be used to detect food borne pathogens all along the supply chain for food, e.g. growing, processing, distribution, retailing, and serving. Currently, bacterium testing represents about 80% of the testing in the food market. Viruses are target with new tests and could become as large an application as bacteria. Examples of food borne viruses include Norwalk-like viruses (Noroviruses) and Rotavirus.

In the present invention, the antibodies or fragments thereof are directed against a viral nucleoprotein and are capable of binding to the viral nucleoprotein. Preferably, the viral nucleoprotein is an influenza viral nucleoprotein and the antibodies and fragments thereof are monoclonal antibodies or fragments thereof directed against influenza virus. A preferred example of influenza viral nucleoprotein is an influenza A viral nucleoprotein and a preferred example of influenza virus is influenza A virus.

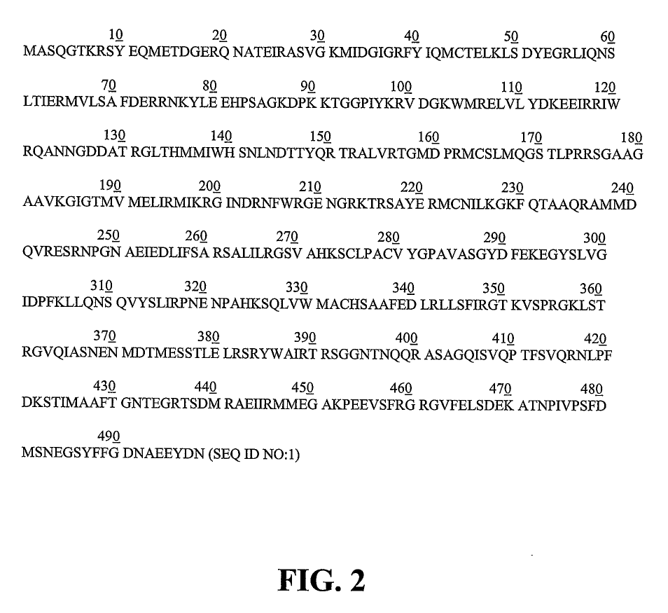

The viral nucleoprotein is a major virion structural protein. The primary function of the nucleoprotein is to encapsidate the viral genome and plays an important role in the viral replication. The influenza A viral nucleoprotein is a polypeptide of 498 amino acids in length, rich in arginine, glycine and serine residues (SEQ ID NO:1, FIG. 2). Nucleoproteins form a superstructure of homo oligomers with K_{d} of ˜200 nM and bind single-stranded RNA (Portela and Digard, J. of General Virology 83, 723-734 (2002)).

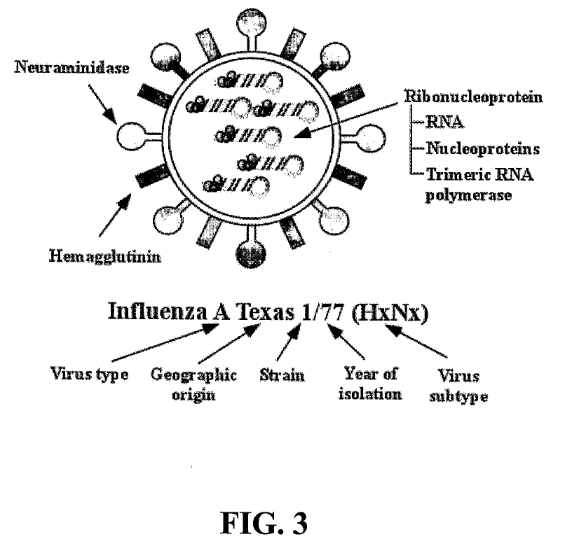

Antibody against influenza A nucleoprotein is selected as it represents a major target antigen in host immune responses. Such antibody recognizes all subtypes of the influenza A nucleoprotein (e.g. all hemagglutinin neuraminidase (HN) categories). Although hemagglutinin and neuraminidase are two viral glycoproteins that are expressed on infected cell surfaces in large quantities (see FIG. 3), they represent only a minority of anti-influenza A virus cytotoxic T lymphocytes target antigens. On the other hand, it has been shown that the nucleoprotein is a major target antigen for the cytotoxic T lymphocytes. Influenza A virus nucleoprotein is an internal virion protein yet present on infected cell surfaces (Yewdell et. al., Proc. Natl. Acad. Sci. USA, 82, 1785-1789 (1985)).

In one embodiment, the antibodies or fragments thereof in the present invention are biotinylated and the second ionophores comprise biotin-modified gramicidin monomers. Addition of streptavidin produces a non-covalent mediated linkage between gramicidin monomers and the antibodies or fragments thereof. The biotinylated antibodies or fragments thereof are therefore linked to the second ionophores through biotin-streptavidin interactions. This technology for the attachment of antibodies or fragments thereof to ionophores relies on a non-covalent complexation or association between biotin and streptavidin.

The biotinylation of Fab is prepared in three stages. First the monoclonal antibody is fragmented using proteolytic enzymes to dimer, F(ab′)₂. Then, the digested fragment is selectively reduced at the disulfide bridge between cysteines, which are at the dimer interface. This results in Fab′ with exposed free sulfhydryl group. Finally, a long chain biotin is ligated to the exposed sulfhydrl group.

In addition to biotin/streptavidin technology, a thiosulfonate-activated ionophore can be used for the direct attachment of antibodies or fragments thereof to ionophores. The thiosulfonate-activated ionophore comprises an ionophore, a spacer group, and an alkylthiosulfonate moiety, wherein the spacer group covalently links the ionophore to the alkylthiosulfonate moiety. The thiosulfonate-activated ionophore technology is described in U.S. Patent Application Publication No. 2005-0250128, the contents of which are incorporated herein by reference.

In one embodiment, the present invention provides methods of detecting a respiratory-related viral nucleoprotein, more specifically, an influenza viral nucleoprotein in a sample with a sensitivity of at least 80%, preferably, 85%, 88%, 90%, 92%, 94%, 96%, 98%, or 99%, and/or a specificity of at least 90%, preferably, 92%, 94%, 96%, 98%, or 99%, and/or an accuracy of at least 90%, preferably, 92%, 94%, 95%, 96%, 97%, 98%, or 99%. The present methods comprise the step of contacting the sample with a biosensor. The sample is either directly applied to the biosensor, or processed or pre-treated prior to the application.

The biosensor suitable for the present methods comprises a membrane and a solid conducting surface, wherein the membrane is attached to the solid conducting surface in a manner such that a reservoir exists between the membrane and the solid conducting surface. The membrane comprises first and second layers each comprising closely packed amphiphilic molecules; a plurality of first and second ionophores located in the first and second layers, respectively, the first and second ionophores such as gramicidin; and a plurality of antibodies or fragments thereof covalently attached to the second ionophores, the antibodies or fragments thereof being capable of binding to the nucleoproteins of respiratory-related viruses, more specifically, the nucleoproteins of an influenza virus. The first ionophores of the membrane are prevented from lateral diffusion in the first layer; and the second ionophores are capable of lateral diffusion within the second layer. The binding of the antibodies or fragments thereof to the viral nucleoprotein causes a change in the relationship between the first and the second ionophores such that the flow of the ions across the membrane via the first and second ionophores is prevented. In this method, reduction of admittance of the membrane corresponds to the presence of a viral nucleoprotein.

In one embodiment, the amphiphilic molecules of the second layer comprise phospholipids, and in one embodiment, the first and second ionophores are gramicidin A.

In another embodiment, the antibodies or fragments thereof are biotinylated antibodies or fragments thereof, and the second ionophores comprise biotin modified gramicidin monomers. With the addition of streptavidin, the biotinylated antibodies or fragments thereof are linked to the second ionophores through biotin-streptavidin interactions.

In yet another embodiment, the influenza viral nucleoprotein is influenza A viral nucleoprotein, and the antibodies or fragments thereof are monoclonal antibodies or fragments thereof directed against influenza A virus.

The present invention also provides a biosensor comprising a membrane and a solid conducting surface, wherein the membrane is attached to the solid conducting surface in a manner such that a reservoir exists between the membrane and the solid conducting surface. The membrane advantageously comprises first and second layers each comprising closely packed amphiphilic molecules; a plurality of first and second ionophores located in the first and second layers, respectively, the first and second ionophores both selected from the group consisting of gramicidin, band three protein, bacteriorhodopsin, proteorhodopsin, mellitin, alamethicin, an alamethicin analogue, porin, tyrocidine, tyrothricin, and valinomycin; and a plurality of antibodies or fragments thereof covalently attached to the second ionophores, the antibodies or fragments thereof being capable of binding to the respiratory-related viral nucleoprotein, more specifically, the influenza viral nucleoprotein. The first ionophores of the membrane are prevented from lateral diffusion in the first layer; and the second ionophores are capable of lateral diffusion within the second layer. The binding of the antibodies or fragments thereof to the respiratory-related viral nucleoprotein, more specifically, the influenza viral nucleoprotein causes a change in the relationship between the first and the second ionophores such that the flow of the ions across the membrane via the first and second ionophores is prevented.

In one embodiment, the amphiphilic molecules of the second layer comprise phospholipids, and in one embodiment, the first and second ionophores are gramicidin A.

In another embodiment, the antibodies or fragments thereof are biotinylated antibodies or fragments thereof, and the second ionophores comprise biotin modified gramicidin monomers. With the addition of streptavidin, the biotinylated antibodies or fragments thereof are linked to the second ionophores through biotin-streptavidin interactions.

In still another embodiment, the influenza viral nucleoprotein is influenza A viral nucleoprotein, and the antibodies or fragments thereof are monoclonal antibodies or fragments thereof directed against influenza A virus.

Depending on the goal to be achieved, the requirement of the sensitivity, specificity, and accuracy can vary. The method of the present invention has at least 80% sensitivity, or at least 90% specificity, or at least 90% accuracy when detecting an influenza viral nucleoprotein. In one embodiment, the method has at least 80%, 85%, 88%, 90%, 92%, 94%, 96%, 98%, or 99% sensitivity. In another embodiment, the method has at least 90%, 92%, 94%, 96%, 98%, or 99% specificity. In yet another embodiment, the method has at least 90%, 92%, 94%, 96%, 98%, or 99% accuracy. In still yet another embodiment, the method of the present invention has at least 80% sensitivity and at least 90% specificity, or at least 80% sensitivity and at least 90% accuracy, or at least 90% specificity and at least 90% accuracy, or at least 80% sensitivity, at least 90% specificity and at least 90% accuracy.

The present invention further provides a biosensor device comprising an array of biosensors described above. Because biosensors measure electrical transduction signals, miniaturization and portability of the device is achievable. The device is useful in that it can measure multiple samples at the same time. In one aspect, the various biosensors can be arranged within a single device containing identical membranes, and are used to detect the same target molecule (analyte) from various samples. In another aspect, the various biosensors can be arranged within a single device containing different membranes, and are used to detect a panel of different analytes either from the same sample or from different samples.

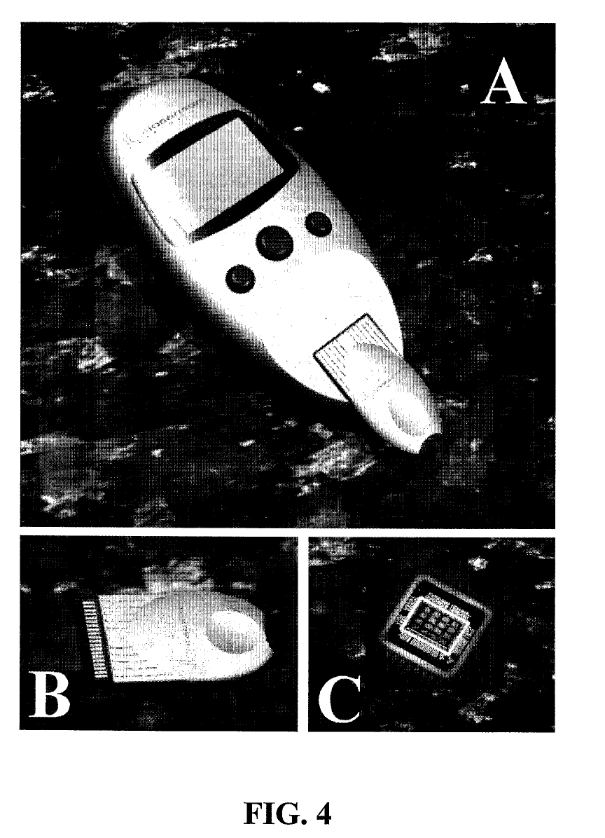

One example of the biosensor device of the present invention is shown in FIG. 4. FIG. 4 is a design for a portable ICS reader and cartridge system suitable for biodefense applications, as well as the consumer and point-of-care markets. Panel A shows a handheld reader connected to a single-use sample test cartridge. Because ICS is based on an electrical transduction mechanism, the necessary detection components are compact. The test cartridge, shown in panel B, houses the microfabricated structure where the ICS chemistry resides and sensing takes place. The ICS biochip would be interfaced with macroscale electrical connections through conventional chip wire bonding as shown in panel C.

The biosensor instrument of the present invention is similar in size and simplicity to the glucose meters used widely today by diabetics. This instrument, as shown in FIG. 4, would function as a reader of microarray chip functionalized with different panels of antibodies or other receptor molecules. These biochips can be embedded within inexpensive disposable microfluidic cartridges useful in measuring very small volumes of environmental samples or bodily fluids. The instrument can provide a direct quantitative result for multiple analytes of interest within minutes.

In order that the invention may be more readily understood, reference is made to the following examples, which are intended to be illustrative of the invention, but are not intended to be limiting in scope.

### Example 1

### Biotinylated Influenza A Antibody Fragment Preparation

Biotinylation of antibody fragments were prepared by a custom antibody processing company (Strategic Biosolution, Newark, Del.) according to the manufacture's standard protocol with slight modification. In brief, starting from 10-20 mg of purified monoclonal antibody against influenza A viral nucleoprotein supplied by Fitzgerald Industries (Concord, Mass.), the antibody fragment dimer, F(ab′)₂, was prepared by digesting the antibody with proteolytic enzyme, pepsin (Biozyme, San Diego, Calif.), at pH 3.5 and 37° C. The resulting dimer was partially purified by dialysis (50 K MWCO) and reduced to a monomer using 2-Mercaptoethylamine-HCl. The reduced monomer has a free sulfhydryl group exposed. Finally, the exposed sulfhydryl group was functionalized using a water-soluble PEO-Iodoacetyl Biotin (Pierce, Rockford, Ill.) according to the manufacture's standard protocol. PEO-Iodoacetyl Biotin has a hydrophilic polyethylene oxide (PEO) spacer arm that gives high water solubility.

### Example 2

### ICS Biosensor Membrane Preparation

The biosensor membranes were prepared based on the procedure described by King et al. (U.S. Pat. No. 5,401,378) with minor modification. The supplied gold deposited slides were incubated in the standard first layer solution (100 mL) in batches. After the incubation at room temperature for 24-hours, the gold slides with the first layer deposited were assembled in 16-sensor well. Then, the second layer and biochemistry components such as influenza A antibodies described in Example 1 were assembled immediately by an automated assembly process using Biomek liquid handler (Beckman Coulter) at room temperature. Individual sensor pads received 15 μL of the standard second layer solution followed by repeated washes with phosphate buffered saline (PBS) buffer solutions. The biochemistry components were then assembled with 100 μL of 84 nM streptavidin followed by 100 μL of 1 μM of the newly prepared biotinylated antibody fragment with extensive PBS washing in between.

### Example 3

### Biosensor Testing

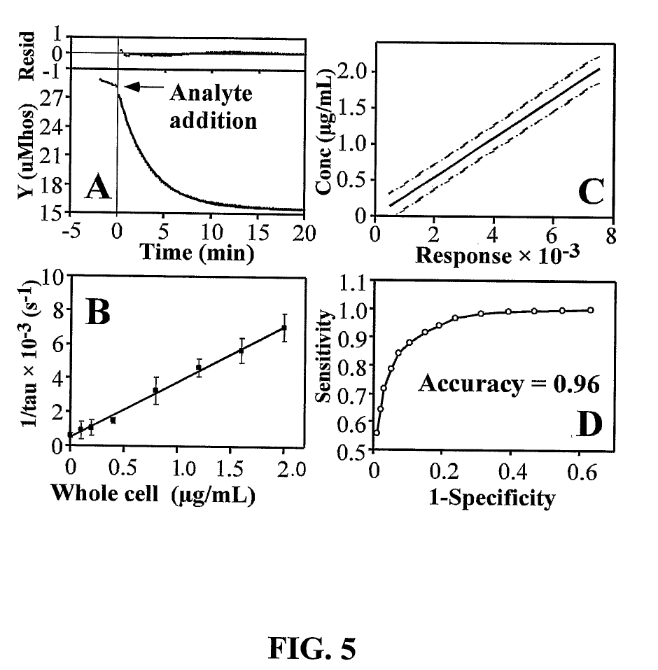

The fully assembled biosensors assembled as described in Example 2 were characterized and assayed using impedance spectroscopy using a series of influenza A viral nucleoprotein dilutions provided by Fitzgerald Industries (Concord, Mass.). The analyte sample was a clarified whole cell sample and contained a high concentration of viral antigens as well as some egg proteins. The analyte dilutions were tested with freshly prepared ICS biosensors for changes in admittance measured at the minimum phase using impedance spectroscopy at 33° C. A series of standard curves were generated and analyzed by linear regression to characterize the status of biosensor performances (see FIG. 5, panel B).

### Example 4

### Influenza A Test

Influenza A test was selected to evaluate the ICS platform as a viral sensor. Influenza A Texas 1/77 from Fitzgerald Industries (Concord, Mass.) was chosen as the initial test strain. Texas 1/77 is the influenza virus type A originated from Texas, strain #1 isolated in 1977 and it recognizes all subtypes (e.g. all hemagglutinin neuraminidase (HN) categories). Inactivated flu analyte was provided by Fitzgerald Industries (Concord, Mass.) as influenza A nucleoprotein whole cell sample.

FIG. 5 shows the ICS test for influenza A, a mild viral pathogen. A series of influenza A viral nucleoprotein dilutions obtained from Fitzgerald Industries (Concord, Mass.) were used as the samples. With the sensor well having a diameter of 5 mm in the present study, about 50-500 uL sample volume can be applied to the sensor well.

FIG. 5, panel A shows a typical ICS influenza A test with a signal exponential decay in admittance upon addition of the analyte. This change in admittance is directly proportional to the concentration of the analyte (FIG. 5, panel B), which gives rise to a reliable quantitative detection method.

### Example 5

### Characterization of Influenza A Test

The ICS biosensor used in the influenza A test described in Example 4 has a high sensitivity, specificity, accuracy and dynamic range, with a low detection limit. To assess the performance characteristics of the influenza A virus antigen test platform, four independent sets of experimental data were analyzed. Analysis of variance (ANOVA) indicates there are some differences between experiments. This difference is attributed to the manual processes involved in this particular assay method. Analysis was carried out using all the data including a random error due to the batch effect. This analysis is conservative as it shows extra large confidence bands for the aggregated data, compared to the much smaller confidence intervals that would result from analysis of individual experimental data sets.

A standard inverse regression (Mendenhall & Sincich, Second Course in Statistics: Regression Analysis (Prentice Hall, Upper Saddle River, N.J., 1996)) was carried out on all experiments to obtain an estimate of the standard deviation for the prediction and the estimate of the regression coefficient. Data from all four runs aggregated together gives a value of 3.3×10⁻³ for the slope with a standard error of 2.2×10⁻⁶ and the residual sum of squares (SSE) of 0.00011. In FIG. 5, panel C, the horizontal axis is the response variable and the vertical axis is the concentration level as predicted by the inverse regression. It shows the 95% confidence band computed with the relevant t quantile; thus, for an observed value of 0.0015, there is a greater than 95% chance that the concentration is strictly higher than zero. This indicates that the detection limit of the system defined by 95% confidence interval is around 0.4 μg/mL.

To further characterize the test platform, a parametric bootstrap (Efron & Tibshirani, An Introduction to the Bootstrap (Chapman and Hall/CRC press LLC, New York, 1998)) of 2000 imaginary subjects (1000 sick patients and 1000 healthy subjects) was simulated. This allowed the generation of an estimate of the Receiver Operating Characteristic (ROC) curve. It was assumed that non-diseased patients have mean concentration of 0.06 μg/mL Fitzgerard protein with a Gaussian distribution and a standard deviation of 0.1 μg/mL Fitzgerard protein. The diseased patients were assumed to have a Gaussian distribution of concentrations with a mean of 0.3 μg/mL and a standard deviation of 0.1 μg/mL. Among these non-diseased patients, the number that would test positive at the cutoff level (0.4 μg/mL) is the false positive rate and among the diseased patients the number of true positives gives the sensitivity of the test.

As shown in FIG. 5, panel D, the ROC curve simulations illustrate an excellent test platform. 0.4 μg/mL cutoff value was used for the ROC curve generation. The ROC curve uses specificity and sensitivity simulated as a function of condition, i.e., cutoff level of 0.4 μg/mL. The accuracy of the test is defined by its capacity to distinguish the group being tested into with and without the presence of influenza A virus antigen. The area under the ROC curve gives an accuracy of 96%.

Table 1 summarizes some of the key analytical properties of the influenza A test. The results show a high linear dose response with an acceptable minimal failure rate of 13%. These performance parameters are expected to improve significantly should the process be automated. The current average of coefficient of variation (CV) (%) is around 17%, which is also expected to improve as the process is optimized and automated.

TABLE 1
ICS Influenza A Test Analytical Properties

| Detection range tested | 0.1 to 2 μg/mL |
|---|---|
| Dose response slope | 3.3 × 10⁻³ per μg/mL |
| Detection limit (95% confidence interval) | 0.4 μg/mL |
| Average % CV (above detection limit) | 17% |
| Failure rate (% dud) | 13% |
| Accuracy (Area under the ROC curve) | 96% |

Specificity, Sensitivity and Area Estimation using
0.4 ug/mL as the cutoff level

| | | | Area | Area | |
|---|---|---|---|---|---|
| | | | estimate | estimate | Area |
| 1-Specificity | Specificity | Sensitivity | low | high | average |
| | | | 0.006 | 0 | 0.003 |
| 0.011 | 0.989 | 0.558 | 0.006 | 0.006 | 0.006 |
| 0.021 | 0.979 | 0.644 | 0.007 | 0.006 | 0.007 |
| 0.031 | 0.969 | 0.718 | 0.017 | 0.015 | 0.016 |
| 0.052 | 0.948 | 0.786 | 0.018 | 0.017 | 0.017 |
| 0.073 | 0.927 | 0.843 | 0.028 | 0.027 | 0.028 |
| 0.105 | 0.895 | 0.879 | 0.041 | 0.040 | 0.040 |
| 0.150 | 0.850 | 0.916 | 0.038 | 0.037 | 0.037 |
| 0.190 | 0.810 | 0.942 | 0.046 | 0.045 | 0.046 |
| 0.238 | 0.762 | 0.967 | 0.073 | 0.072 | 0.072 |
| 0.312 | 0.688 | 0.984 | 0.078 | 0.078 | 0.078 |
| 0.391 | 0.609 | 0.991 | 0.075 | 0.074 | 0.074 |
| 0.466 | 0.534 | 0.994 | 0.083 | 0.083 | 0.083 |
| 0.549 | 0.451 | 0.997 | 0.080 | 0.080 | 0.080 |
| 0.629 | 0.371 | 0.999 | 0.371 | 0.371 | 0.371 |
| | | | 0 | 0 | 0 |

| | | | |
|---|---|---|---|
| Sum of Area | 0.967 | 0.949 | 0.958 |

Accuracy was determined by estimating area under ROC curve using trapezoidal rule (Hanley, J., and McNeil, B. 1982. The meaning and use of the area under a Receiver Operating Characteristic (ROC) curve. Radiology 143: 29-36).

### Example 6

### Comparative Sensitivity Analysis of ICS vs. ELISA-based Detection of Influenza A Virus

Takara Influenza A ELISA Kit (catalog number #MK120, Kyoto, Japan) and influenza A nucleoprotein antigen sample from Fitzgerald Industries (Concord, Mass.) were used. Such antigen sample is a crude viral lysis preparation, which also contains chicken egg proteins. ICS biosensor was assembled as described in Example 2.

Influenza A quantitative ELISA kit from Takara is a solid phase EIA-based sandwich method that utilizes two antibodies to influenza A virus by two step procedure. The experimental protocol for creating standard curve using the positive control sample provided by the kit was followed as detailed in the manual. The standard curve was generated by using (in quadruplet) the positive control sample in dilution ranging 20, 10, 5, 2.5, 1.25, 0.625, and 0 HA unit/ml. HA is a unit of influenza virus by the method of erythrocyte aggregation. One HA unit equals the quantity of virus needed to aggregate erythrocyte completely with no dilution. One unit, which was used in this study, equals the quantity of virus antigen of one HA. This ELISA kit was then used to quantitatively determine the nucleoprotein antigen in Fitzgerald antigen sample (n=5) in dilution ranging 2, 1.6, 1.2, 0.8, 0.4, 0.2, 0.1, and 0 on the basis of total protein determined in μg/ml. Using linear regression from the standard curve, influenza A nucleoprotein antigen concentration (in HA unit/ml) was determined. ICS influenza A test as described in Example 4 was also conducted using the same Fitzgerald sample with exact the same dilutions as the ones used in ELISA study.

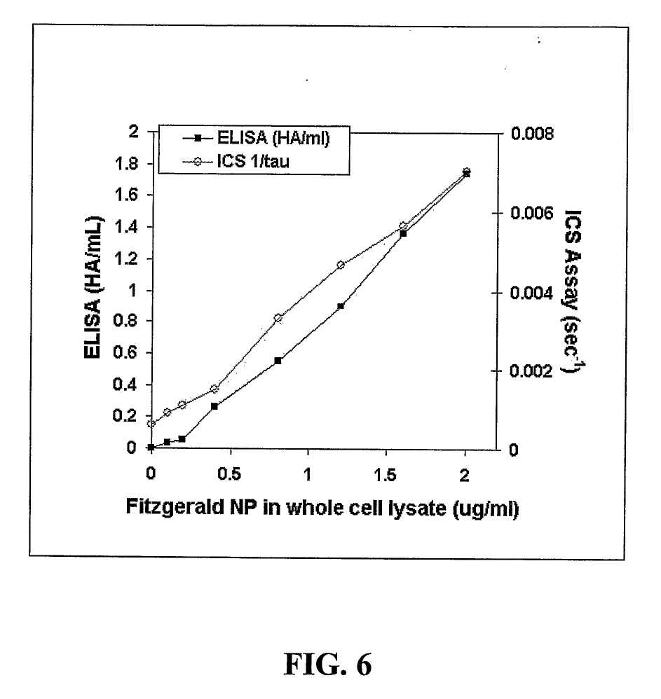

As illustrated in FIG. 6, a direct comparison and correlation of ICS™ with ELISA kit (Takara Miru-Madison, Wis.) for influenza A test showed a comparable detection limits while ICS uses only about 40-400× less antibody. It is known that ELISA plastic plates have a finite binding capacity in the range of 50 to 500 ng per well when added as 50 μL volumes (The ELISA Guidebook, Ed., JR Crowther, Method in Molecular Biology, vol. 149, 2001, Humana Press, NJ USA). ICS uses 250 ng/mL of Fabs, 25 ng/mL of MSL4xB, and 200 ng/mL of gA5xB whereas ELISA uses 1000 to 10,000 ng/mL antibody concentration to saturate the plate. MSL4xB and gA5xB are biotinylated membrane spanning lipid with tetraethyleneglycol linker and biotinylated gramicidin A with pentaethyleneglycol linker, respectively. These two molecules are used to attach antibody fragments (Fabs) to the ICS™ biosensor platform.

In three repeated measurements, ICS™ and ELISA showed detection limit of 2.2 μg/mL and 1.2 μg/mL Fitzgerald influenza A, respectively. The published sensitivity for the Takara ELISA kit is 0.4 HA unit which translates to 2.9 μg/mL Fitzgerald influenza A virus total protein concentration.

In terms of use and time spent to complete the assay, ICS method has a significant time advantage and ease of use over ELISA assay. In particular, it usually takes between 4-5 hours to complete ELISA assay; however, it only takes 2 hours for ICS assay.

### Example 7

### Comparative Sensitivity Analysis of ICS vs. BD Directigen Influenza A Virus Test Kit

Directigen™ Flu A test kit (Beckton-Dickinson, BD) was selected as a commercially available benchmark test as it is currently used as industry standard rapid screening test kit for influenza A virus. The test analyte, supplied by BD Immuno Diagnostics group from BD Diagnostic Systems, was influenza A Virus (H1N1) (total protein, 1.3 mg/mL) from Allantoic fluid of 10 day old embryonated eggs inoculated with Flu A/New Calcdonia/20/99, purified by ultracentrifugation using 30-60% sucrose gradient and inactivated by 0.005% Merthiolate.

In order to quantify the BD test strip results for comparison purpose, the BD test strips were taken apart and scanned for image analysis using Photoshop. Histogram median values from each scanned images were sampled from selected ellipses within the area of interest. For each test, three readings were collected from the ellipses moved to the following three different areas: 1) the outside circle region; 2) the triangular “positive reaction” area; and 3) the inside dot area, with the reading at the third area signifies that the reaction worked. These three readings were then averaged with repeats and reported. A positive reaction value is the ratio of the reaction area divided by the surrounding outside circle region. If there is no reaction the ratio will be 1.0. The lower the ratio is, the stronger the reaction is.

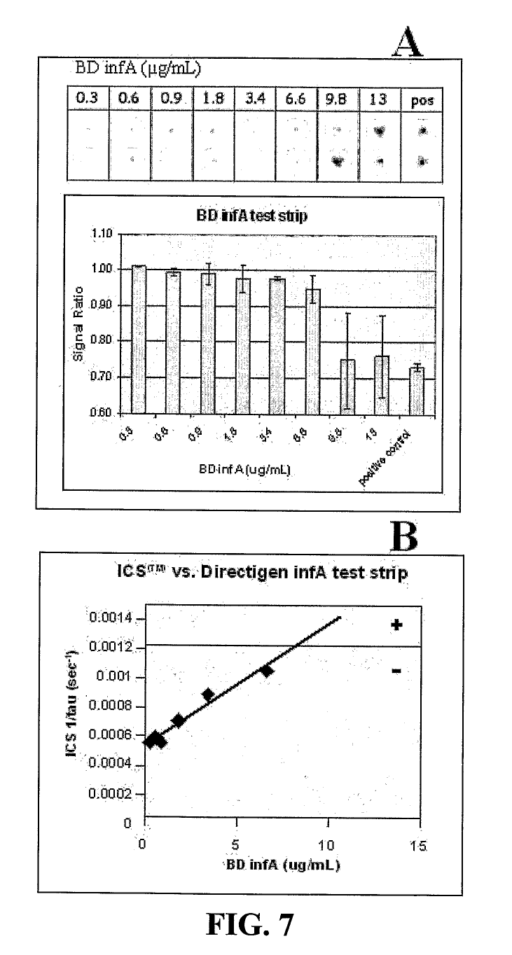

FIG. 7 shows the BD test strip (panel A) and ICS test (panel B) results for influenza A test. In Panel A, top view demonstrates a series of images of BD test strip results with different dilutions, and bottom view demonstrates signal ratio of BD test image analysis at different concentrations. Signal ratio above 0.9 indicates negative results while signal ratio below 0.9 indicates positive test results. The ICS test results presented in Panel B demonstrates linearity in the signal output in the BD test strip negative concentration region indicated by (−) area. ICS influenza A test showed detection limit of 0.5 μg/mL BD influenza A virus. The BD Directigen™ test kit showed the detection limit of between 6.6 and 9.8 μg/mL.

### Example 8

### Receiver Operating Characteristics (ROC) Curve Analysis of ICS Influenza A Test System

The ROC curve is frequently used to evaluate the accuracy of medical diagnostic tests. An ROC curve plots the sensitivity of a diagnostic test (on the y-axis) over all possible false positive rates (the x-axis). The Area under the Curve (AUC) ranging between 0 and 1 is a measure of accuracy of a diagnostic test. In particular, larger values indicate better accuracy. The AUC statistic can be interpreted as the probability that the test result from a diseased individual is more indicative of disease than that from a non-diseased individual.

A “gold standard” is necessary to define the true condition status of the patient. Clinical data consisting of diagnoses of a population of patients often serves this purpose. The gold standard must be able to produce a dichotomous outcome (diseased or not diseased) for each test case. Clinical data, however, are unavailable to serve as a gold standard for the diagnostic data in the present study. One approach of dealing with the lack of clinical data is to substitute it with some independently measured factor that indicates true disease status. This approach is often used when the clinical outcome is on a continuous rather than binary scale (Obuchowski, et al., Clin Chem 50(7), 1118-25 (2004)), such as blood glucose level as an indicator of hypoglycemia (Pitzer, et al., Diabetes Care 24(5), 881-5 (2001)). In this case, a threshold value must be chosen to separate the test subjects into diseased and non-diseased populations. The arbitrarily chosen threshold level can introduce bias, however, and ideally should be chosen to have some clinical significance.

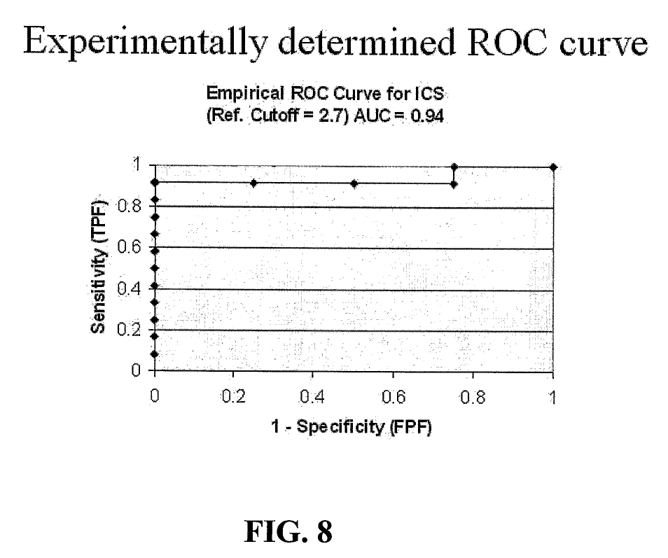

The ROC curve analysis shown in FIG. 8 indicates that the ICS™ FluA test gives 92% sensitivity at 100% specificity and overall accuracy of 94%. The ROC curve was generated using an Excel macro. The macro was used to create ROC curves as described below for cases when there is a continuous-scale measurement serving as the gold standard. Using the macro, the diagnostic and gold standard pairs of values for each test case was identified, and then a threshold value for the gold standard data, where the true disease state of the test case is considered positive, was selected. The results generated by the macro are compiled in a data table for the specificity and sensitivity values at each threshold decision point, and the area under the curve (AUC) is also calculated.

To produce the ROC curve shown in FIG. 8, a diagnostic test measurement value and gold standard measurement were first obtained for each test case. Then, the data were sorted by increasing diagnostic test value, and a threshold was set for the gold standard values as described above (this value was held constant throughout, and provided an evaluation of the true disease status of each test case).

The threshold for the diagnostic test values was then set such that all values greater than or equal to the lowest diagnostic value in the data set is considered a positive decision (or alternatively a negative decision if lower values are considered more indicative of disease status) by the diagnostic. For each test case, the diagnostic decision was then compared to its corresponding gold standard value to determine whether the diagnostic correctly predicted the true disease status, and the sensitivity and specificity for the entire data set were calculated for that diagnostic threshold value. The diagnostic test value threshold was then set such that all values greater than or equal to the second highest diagnostic test value was considered positive (or negative), and sensitivity and specificity were again calculated for the entire data set. The threshold was thus successively set at every possible diagnostic decision threshold. For every such decision threshold, the sensitivity was plotted on the y-axis against (1-specificity) (i.e., the false positive rate) on the x-axis in FIG. 8.

The results show that the ICS™ FluA test is a very accurate and sensitive test with low false positive rate. In particular, the ICS™ FluA test is shown to give 92% sensitivity at 100% specificity and overall accuracy of 94%.

It will be obvious to those skilled in the art that various changes may be made without departing from the scope of the invention, which is not to be considered limited to what is described in the specification.

1. A method of detecting respiratory-related viruses in a sample, comprising the steps of:
contacting the sample with a biosensor, wherein the biosensor comprises a membrane and a solid conducting surface, wherein the membrane is attached to the solid conducting surface in a manner such that a reservoir exists between the membrane and the solid conducting surface, wherein the membrane comprises:
first and second layers each comprising closely packed amphiphilic molecules;
a plurality of first and second ionophores located in the first and second layers, respectively, the first and second ionophores both selected from the group consisting of gramicidin, band three protein, bacteriorhodopsin, proteorhodopsin, mellitin, alamethicin, an alamethicin analogue, porin, tyrocidine, tyrothricin, and valinomycin; and
a plurality of antibodies or fragments thereof covalently attached to the second ionophores, the antibodies or fragments thereof being capable of binding to nucleoproteins of the respiratory-related viruses;
wherein the first ionophores are prevented from lateral diffusion in the first layer; and the second ionophores are capable of lateral diffusion within the second layer;
whereby the binding of the antibodies or fragments thereof to the nucleoproteins of the respiratory-related viruses causes a change in the relationship between the first and the second ionophores such that the flow of the ions across the membrane via the first and second ionophores is prevented;
wherein reduction of admittance of the membrane corresponds to the presence of respiratory-related viruses.

2. The method according to claim 1, wherein said respiratory-related virus is an influenza virus.

3. The method according to claim 2, wherein the influenza virus is influenza A virus.

4. The method according to claim 1, wherein said method has a sensitivity of at least 80%, 85%, 88%, 90%, 92%, 94%, 96%, 98%, or 99%.

5. The method according to claim 1, wherein said method has a specificity of at least 90%.

6. (canceled)

7. The method according to claim 1, wherein said method has an accuracy of at least 90%.

8. (canceled)

9. The method according to claim 1, wherein said method has a sensitivity of at least 80% and an accuracy of at least 90%.

10. The method according to claim 1, wherein said method has a specificity of at least 90% and an accuracy of at least 90%.

11. The method according to claim 1, wherein said method has a sensitivity of at least 80% and a specificity of at least 90%.

12. The method according to claim 1, wherein said method has a sensitivity of at least 80%, a specificity of at least 90% and an accuracy of at least 90%.

13. The method according to claim 1, wherein said method has a detection limit of at least 0.5 μg/mL.

14. (canceled)

15. The method according to claim 1, wherein the sample is a body sample is selected from the group consisting of blood, serum, sweat, tears, urine, saliva, throat swabs, nasopharyngeal aspirates, smears, bile, gastrointestinal secretions, lymph, organ aspirates and biopsies.

16. (canceled)

17. The method according to claim 1, wherein the sample is a non-body sample is selected from the group consisting of culture medium, water, saline, organic acids, buffers, soil, food, beverages, powders, building and room surfaces.

18. The method according to claim 1, wherein the amphiphilic molecules of the second layer comprise phospholipids.

19. The method according to claim 1, wherein the first and second ionophores are gramicidin A.

20. The method according to claim 1, wherein the antibodies or fragments thereof are biotinylated antibodies or fragments thereof.

21. (canceled)

22. The method according to claim 3, wherein the antibodies or fragments thereof are monoclonal antibodies or fragments thereof directed against influenza A virus.

23. A biosensor comprising a membrane and a solid conducting surface, wherein the membrane is attached to the solid conducting surface in a manner such that a reservoir exists between the membrane and the solid conducting surface, wherein the membrane comprises:
first and second layers each comprising closely packed amphiphilic molecules;
a plurality of first and second ionophores located in the first and second layers, respectively, the first and second ionophores both selected from the group consisting of gramicidin, band three protein, bacteriorhodopsin, proteorhodopsin, mellitin, alamethicin, an alamethicin analogue, porin, tyrocidine, tyrothricin, and valinomycin; and
a plurality of monoclonal antibodies or fragments thereof directed against respiratory-related viruses and covalently attached to the second ionophores, the antibodies or fragments thereof being capable of binding to nucleoproteins of respiratory-related viruses;
wherein the first ionophores are prevented from lateral diffusion in the first layer; and the second ionophores are capable of lateral diffusion within the second layer;
whereby the binding of the antibodies or fragments thereof to the nucleoproteins of the respiratory-related viruses causes a change in the relationship between the first and the second ionophores such that the flow of the ions across the membrane via the first and second ionophores is prevented.

24. The biosensor according to claim 23, wherein said respiratory-related virus is an influenza virus.

25. The biosensor according to claim 24, wherein the influenza virus is influenza A virus.

26. The biosensor according to claim 23, wherein the amphiphilic molecules of the second layer comprise phospholipids.

27. The biosensor according to claim 23, wherein the first and second ionophores are gramicidin A.

28. The biosensor according to claim 23, wherein the antibodies or fragments thereof are biotinylated antibodies or fragments thereof.

29. (canceled)

30. The biosensor according to claim 25, wherein the monoclonal antibodies or fragments thereof are directed against influenza A virus.

31. A handheld biosensor device comprising an array of biosensors according claim 23.
